(19) United States
(12) Patent Application Publication
(10) Pub. No.: US 2013/0237809 A1
(43) Pub. Date: Sep. 12, 2013

(54) POSITION DETECTING APPARATUS OF CAPSULE ENDOSCOPE AND CAPSULE ENDOSCOPE SYSTEM

(71) Applicant: OLYMPUS MEDICAL SYSTEMS CORP., Tokyo (JP)

(72) Inventor: Jun HASEGAWA, Tokyo (JP)

(73) Assignee: OLYMPUS MEDICAL SYSTEMS CORP., Tokyo (JP)

(21) Appl. No.: 13/771,126

(22) Filed: Feb. 20, 2013

Related U.S. Application Data
(63) Continuation of application No. PCT/JP2012/052758, filed on Feb. 7, 2012.

(30) Foreign Application Priority Data
Mar. 2, 2011 (JP) 2011-045684

(51) Int. Cl. A61B 1/04; A61B 1/00; A61B 5/00; A61B 5/06

(52) U.S. Cl. CPC A61B 1/041; A61B 5/061; A61B 1/00016; A61B 5/72; A61B 5/742
USPC 600/424

(57) ABSTRACT

A position detecting apparatus of a capsule endoscope includes a receiving antenna unit for receiving, by a plurality of receiving antennas, a wireless signal transmitted from a capsule endoscope within a subject, a storage unit for storing theoretical electric field strengths of the wireless signal received by the receiving antennas depending on positions or positions and orientations of the capsule endoscope in the subject, a comparing unit for comparing specified values that are calculated from a difference between received electric field strengths of the wireless signal received by the receiving antennas and the theoretical electric field strengths stored in the storage unit, and a determination unit for determining a position or a position and orientation of the capsule endoscope where image data has been taken, based on a comparison result by the comparing unit.

### CROSS REFERENCES TO RELATED APPLICATIONS

This application is a continuation of PCT international application Ser. No. PCT/JP2012/052758 filed on Feb. 7, 2012 which designates the United States, incorporated herein by reference, and which claims the benefit of priority from Japanese Patent Applications No. 2011-045684, filed on Mar. 2, 2011, incorporated herein by reference.

### BACKGROUND OF THE INVENTION

1. Field of the Invention

The present invention relates to a position detecting apparatus and a capsule endoscope system for receiving a wireless signal transmitted from a capsule endoscope within a subject by a receiving device that is disposed outside the subject and detecting a position of the capsule endoscope based on the received wireless signal.

2. Description of the Related Art

Conventionally, in the field of endoscopes, known are capsule endoscopes in which an imaging function, a radio communication function, and the like are embedded in a capsule-shaped casing formed in a size that is able to introduced into a gastrointestinal tract of a subject such as a patient. After being swallowed from the subject's mouth, the capsule endoscope moves within the subject such as the gastrointestinal tract by peristalsis motion and the like. Then, it sequentially captures the inside of the subject to generate image data and sequentially transmits the image data in a wireless manner.

The image data wirelessly transmitted from the capsule endoscope in such a way is received at the receiving device provided outside the subject. The image data received by the receiving device is stored in a memory embedded in the receiving device. Upon completion of the examination, the image data accumulated in the memory of the receiving device is inputted to the image display device. The observer such as a doctor or a nurse observes the internal organ displayed on the image display device and the subject is diagnosed.

Because the capsule endoscope moves by the peristalsis motion in the body cavity, it is necessary to correctly recognize at which position in the body cavity the image data transmitted by the capsule endoscope is taken.

In this regard, a capsule endoscope is disclosed that receives the electromagnetic wave transmitted by the capsule endoscope by a plurality of receiving antennas outside the body cavity and estimates the position and orientation of the capsule endoscope by using a Gaussian Newton method based on the received strength of a plurality of received wireless signals (Japanese Laid-open Patent Publication No. 2007-000608).

Further, a capsule endoscope is disclosed that provides a sensor for collecting intra-subject information and recognizes the position and the like of the capsule endoscope that is inside the subject based on the information collected by the sensor (Japanese National Publication of International Patent Application No. 2010-524557).

### SUMMARY OF THE INVENTION

A position detecting apparatus of a capsule endoscope according to one aspect of the present invention includes: a receiving antenna unit for receiving, by a plurality of receiving antennas, a wireless signal transmitted from a capsule endoscope within a subject; a storage unit for storing, in advance, information indicating a first position of the capsule endoscope within the subject and information indicating a first theoretical electric field strength of the wireless signal received by each of the antennas depending on the first position, in such a manner that the information of the first position is associated with the information of the first theoretical electric field strength, and for storing, in advance, information indicating a second position of the capsule endoscope within the subject and information indicating a second theoretical electric field strength of the wireless signal received by each of the antennas depending on the second position, in such a manner that the information of the second position is associated with the information of the second theoretical electric field strength; an electric field strength comparing unit for comparing a received electric field strength of the wireless signal received by each of the receiving antennas with the first theoretical electric field strength and for comparing the received electric field strength with the second theoretical electric field strength; and a position determination unit for determining either one of the first position and the second position, as a position of the capsule endoscope where image data has been taken, based on a comparison result by the electric field strength comparing unit.

A capsule endoscope system according to one aspect of the present invention includes: a capsule endoscope for obtaining image data of an inside of a subject; the position detecting apparatus for receiving the image data transmitted from the capsule endoscope and estimating a position and orientation of the capsule endoscope where the received image data has been taken; and an image display unit for obtaining the image data and position information of the image data from the receiving antenna and the position detecting apparatus and for displaying the obtained image data and position information.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating a schematic configuration of a capsule endoscope system using a receiving device according to a first embodiment of the present invention;

FIG. 2 is a cross-sectional view illustrating a schematic internal configuration of the capsule endoscope;

FIG. 3 is a block diagram illustrating a schematic configuration of the receiving device according to the first embodiment of the present invention;

FIG. 4A is a schematic diagram for explaining position detection of the capsule endoscope;

FIG. 4B is a schematic diagram in which a region of FIG. 4A is divided into four regions in each of x, y, and z directions;

FIG. 5 is a schematic view illustrating electromagnetic field components in a arbitrary position with respect to an antenna (using a circle coil) of the capsule endoscope;

FIG. 6 is a schematic view illustrating that the electromagnetic field attenuates when propagating in a medium;

FIG. 7 is a schematic view illustrating a relationship between an electric field generated by the capsule endoscope and a direction of one of receiving antennas of a receiving antenna unit;

FIG. 8A is a schematic diagram in which the region where the capsule endoscope is present is divided into three regions in each of the x, y, and z directions;

FIG. 8B is a schematic diagram in which one of the regions of FIG. 8A is further divided into three regions in each of the x, y, and z directions;

FIG. 9 is a block diagram illustrating a schematic configuration of a receiving device according to a third embodiment of the present invention;

FIG. 10 is a flowchart illustrating an outline of a trajectory calculation process performed by a trajectory calculation unit;

FIG. 11 is a schematic view illustrating a plurality of candidate positions that have been position-estimated for a plurality of image data taken at a previous timing and a subsequent timing;

FIG. 12 is a flowchart of the trajectory calculation process;

FIG. 13A is a display example on an image display device that shows a trajectory of the capsule endoscope within a subject calculated by the receiving device of the third embodiment; and

FIG. 13B is a display example on the image display device that shows a trajectory of the capsule endoscope within the subject calculated by the receiving device of the third embodiment.

### DETAILED DESCRIPTION OF THE PREFERRED EMBODIMENTS

A position detecting apparatus and a capsule endoscope system according to the embodiment of the present invention will be described below by referring to the drawings. It is noted that, in the following description, while a capsule endoscope system including a capsule endoscope that is introduced in a subject's body and captures in-vivo images of the subject will be exemplified as an example of the position detecting apparatus and the capsule endoscope system according to the present invention, the present invention is not limited to the embodiment.

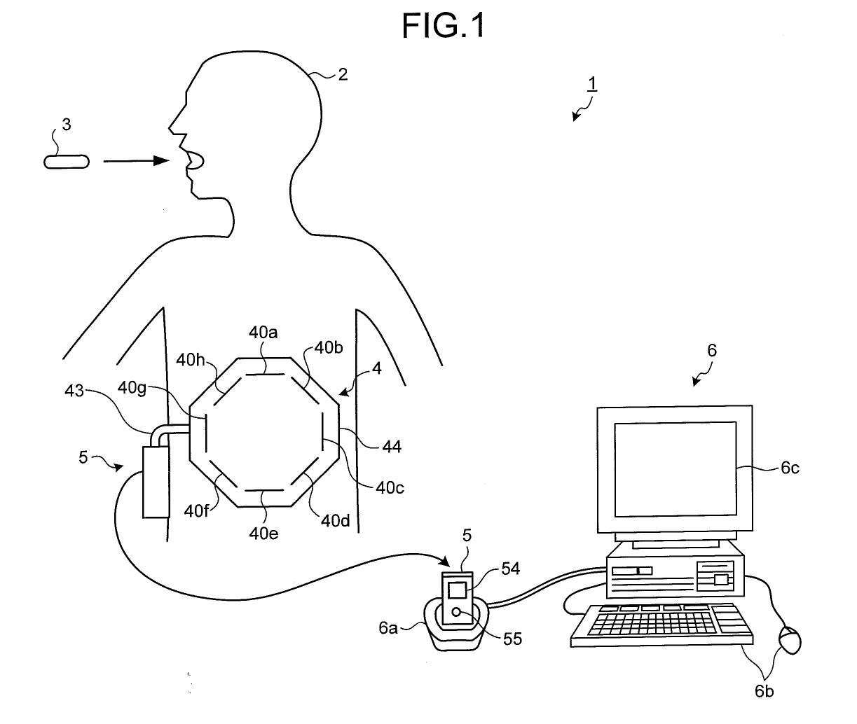

FIG. 1 is a schematic diagram illustrating a schematic configuration of a capsule endoscope system 1 with a position detecting apparatus according to a first embodiment of the present invention. As illustrated in FIG. 1, the capsule endoscope system 1 includes a capsule endoscope 3 that captures in-vivo images of a subject 2, a receiving device 5 that receives a wireless signal transmitted by the capsule endoscope 3 introduced into the subject 2 and receives a capturing position of the image data of the inside of the subject 2 captured by the capsule endoscope 3, and an image display device 6 that displays an image corresponding to the image data of the inside of the subject 2 captured by the capsule endoscope 3.

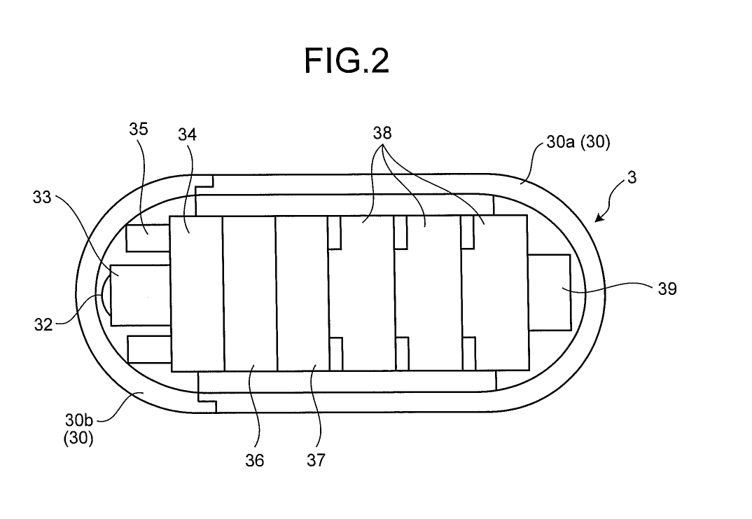

FIG. 2 is a cross-sectional view illustrating the schematic internal configuration of the capsule endoscope 3. As illustrated in FIG. 2, the capsule endoscope 3 is contained in a capsule container 30 (casing) having an approximately cylindrical or semi-ellipse sphere container 30a, one end of which is semi-sphere dome shape and the other end of which is opened, and an semi-sphere optical dome 30b that is fitted into the opening of the container 30a to seal the container 30a in a watertight manner. The capsule container 30 (30a, 30b) is, for example, of a size that the subject 2 can swallow. Further, in the first embodiment, at least the optical dome 30b is formed with a transparent material.

Further, the capsule endoscope 3 includes an objective lens 32 for forming an image of the light entered through the optical dome 30b, a lens frame 33 by which the objective lens 32 is attached, an imaging unit 34 for converting an optical signal entered from the objective lens 32 into an electrical signal to form a capturing signal, a lighting unit 35 for lighting the inside of the subject 2 at the imaging, a circuit board 36 on which a processing circuit and the like for driving the imaging unit 34 and the lighting unit 35, respectively, and generating an image signal from the imaging signal entered from the imaging unit 34 are formed, a transceiving circuit 37 for transmitting the image signal and receiving a signal from the receiving device 5 and the like that are disposed outside the body cavity, and a plurality of button batteries 38 for supplying power to respective function units described above.

The capsule endoscope 3 passes through the esophagus in the subject 2 after swallowed into the subject and moves inside the body cavity by the peristalsis motion of the gastrointestinal tract cavity. The capsule endoscope 3 sequentially captures the inside of the body cavity of the subject 2 at a short interval of time such as an interval of 0.5 second, while moving inside the body cavity, and generates the image data of the inside of the captured subject 2 to sequentially transmits it to the receiving device 5. In the first embodiment, although it is possible to perform the position estimation process by the image signal of the image data taken by the imaging unit 34 of the capsule endoscope 3, it is preferable to generate a transmission signal including the captured image signal and the received strength detecting signal for the position detection of the capsule endoscope 3 and perform the position detection process by using the received strength detection signal whose received strength can be easily detected.

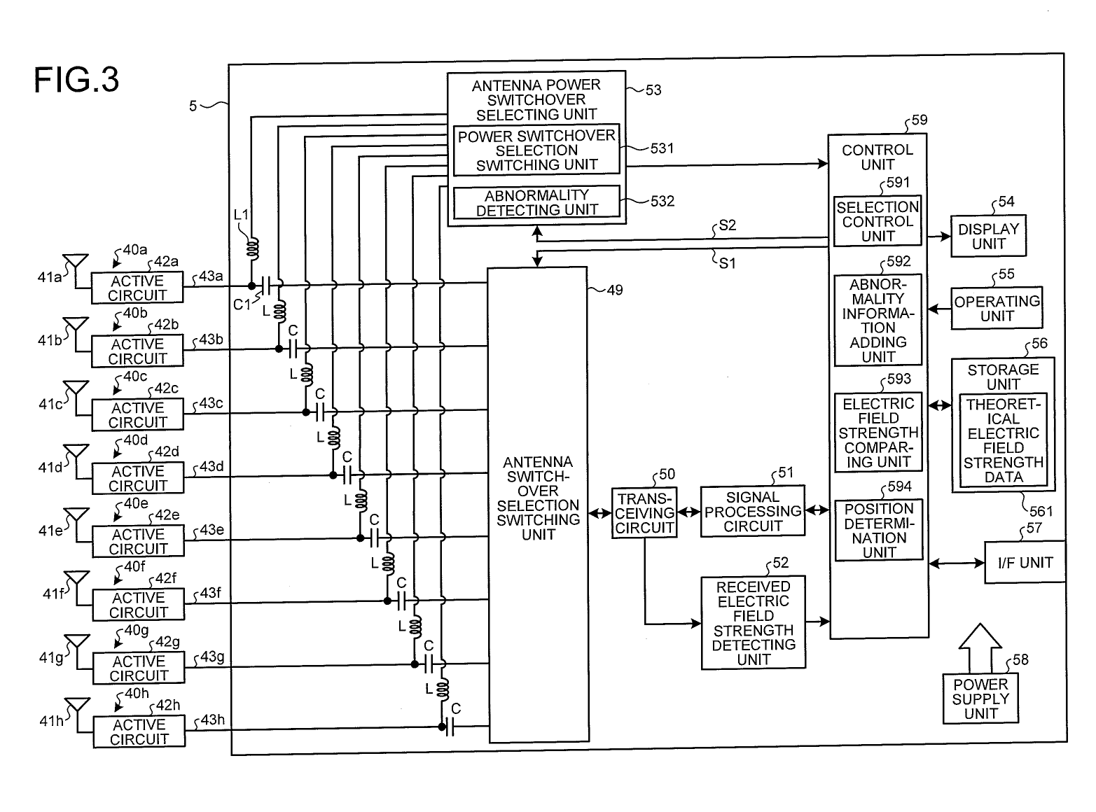

The position detecting apparatus includes a sheet-shaped receiving antenna unit 4 on which a plurality of receiving antennas 40 (40a, 40b, 40c, 40d, 40e, 40f, 40g, 40h) are provided and the receiving device 5. The receiving device 5 is connected to the receiving antenna unit 4 by an antenna cable 43. The receiving device 5 receives the wireless signal transmitted from the capsule endoscope 3 through each of the receiving antennas 40a to 40h. The receiving device 5 detects the received electric field strength of the wireless signal received from the capsule endoscope 3 for each of the receiving antennas 40a to 40h and obtains the image data inside the subject 2 based on the received wireless signal. The receiving device 5 associates the received electric field strength information, the time information indicating the time, and so on with the received image data for each of the receiving antennas 40a to 40h and stores them in a storage unit (see FIG. 3) described later.

While the capturing is being performed by the capsule endoscope 3, the receiving device 5 is carried by the subject 2, for example, after it is introduced into the subject 2 from its mouse and passes through the gastrointestinal tract before excreted from the subject 2. Upon the completion of the examination by the capsule endoscope 3, the receiving device 5 is removed from the subject 2 and connected to the image display device 6 for transferring the information such as the image data received from the capsule endoscope 3.

The receiving antennas 40a to 40h are disposed at specified positions on a sheet 44. For example, the specified positions correspond to organs of the subject 2 along a path of the endoscope 3 when the receiving antenna unit 4 is attached to the subject 2. It is noted that the arrangement of the receiving antennas 40a to 40h may be changed according to the purpose such as the examination, the diagnosis, and the like. Although eight receiving antennas are used in the present embodiment, it is not necessary to limit the number of the receiving antennas to eight, and the number may be less than eight or greater than eight.

The image display device 6 is configured with a workstation or a personal computer having a monitor unit 6c such as a liquid crystal display. The image display device 6 displays the image corresponding to the image data of the inside of the subject 2 obtained through the receiving device 5. A cradle 6a and an operation input device 6b such as a keyboard, a mouse, and so on are connected to the image display device 6. When the receiving device 5 is attached, the cradle 6a acquires the image data from the memory of the receiving device 5, the received electric field strength information associated with the image data, and the associated information such as the time information and the identification information of the capsule endoscope 3, and the like, and transfers the acquired various sorts of information to the image display device 6. The operation input device 6b accepts the user input. This allows the user to observe the organism part such as the esophagus, the stomach, the small intestine, the large intestine, and so on and diagnose the subject 2 while operating the operation input device 6b and watching the image of the inside of the subject 2 displayed at the image display device 6.

Next, the configuration of the receiving device 5 illustrated in FIG. 1 will be described. FIG. 3 is a block diagram illustrating the configuration of the receiving device 5 illustrated in FIG. 1.

As illustrated in FIG. 3, the receiving device 5 has the receiving antennas 40a to 40h described above, an antenna switchover selection switching unit 49 for alternatively switching the receiving antennas 40a to 40h, a transceiving circuit 50 for applying the process such as demodulation to the wireless signal received via any one of the receiving antennas 40a to 40h selected by the antenna switchover selection switching unit 49, a signal processing circuit 51 for performing the signal processing for extracting the image data and the like from the wireless signal outputted from the transceiving circuit 50, a received electric field strength detecting unit 52 for detecting the received electric field strength based on the strength of the wireless signal outputted from the transceiving circuit 50, an antenna power switchover selecting unit 53 for alternatively switching the receiving antennas 40a to 40h to supply power to any one of the receiving antennas 40a to 40h, a display unit 54 for displaying the image corresponding to the image data received from the capsule endoscope 3, an operating unit 55 for making an instruction operation, a storage unit 56 for storing various sorts of information including the image data received from the capsule endoscope 3, an I/F unit 57 for communicating with the image display device 6 interactively via the cradle 6a, a power supply unit 58 for supplying power to respective units of the receiving device 5, and a control unit 59 for controlling the operation of the receiving device 5.

The receiving antenna 40a has an antenna unit 41a, an active circuit 42a, and an antenna cable 43a. The antenna unit 41a is configured with, for example, an open antenna or a loop antenna, and receives the wireless signal transmitted from the capsule endoscope 3. The active circuit 42a is connected to the antenna unit 41a for matching the impedance of the antenna unit 41a, amplifying and/or attenuating the received wireless signal, and so on. The antenna cable 43a is configured with a coaxial cable, one end of which is electrically connected to the active circuit 42a and the other end of which is electrically connected to the antenna switchover selection switching unit 49 and the antenna power switchover selecting unit 53, respectively. The antenna cable 43a transmits the wireless signal received by the antenna unit 41a to the receiving device 5 and transfers the power supplied from the receiving device 5 to the active circuit 42a. It is noted that, since each of the receiving antennas 40b to 40h has the same configuration as the receiving antenna 40a, the description thereof is omitted.

The antenna switchover selection switching unit 49 is configured with a mechanical switch, a semiconductor switch, and the like. The antenna switchover selection switching unit 49 is electrically connected to each of the receiving antennas 40a to 40h via a capacitor C1. When a switching signal S1 for switching the receiving antennas 40a to 40h for receiving the wireless signal from the control unit 59 is inputted, the antenna switchover selection switching unit 49 selects the receiving antenna 40 instructed by the switching signal S1, and outputs the wireless signal received via the selected receiving antennas 40a to 40h to the transceiving circuit 50. It is noted that each capacitor connected to each of the receiving antennas 40a to 40h has the same capacitance as the capacitor C1.

The transceiving circuit 50 applies a specified operation such as demodulation, amplification, and so on to the wireless signal received via the receiving antenna 40 (40a to 40h) selected by the antenna switchover selection switching unit 49 and outputs it to the signal processing circuit 51 and the received electric field strength detecting unit 52, respectively.

The signal processing circuit 51 extracts the image data from the wireless signal inputted from the transceiving circuit 50, and applies a specified process such as various sorts of image processing, A/D conversion processing, and so on to the extracted image data and outputs it to the control unit 59.

The received electric field strength detecting unit 52 detects the received electric field strength depending on the strength of the wireless signal inputted from the transceiving circuit 50 and outputs, to the control unit 59, the received electric field strength signal (RSSI: Received Signal Strength Indicator) corresponding to the strength of the wireless signal inputted from the transceiving circuit 50.

The antenna power switchover selecting unit 53 is electrically connected to each of the receiving antennas 40a to 40h via a coil L1. The antenna power switchover selecting unit 53 supplies power to the receiving antennas 40a to 40h selected by the antenna switchover selection switching unit 49 via the antenna cable 43 (43a to 43h). The antenna power switchover selecting unit 53 has a power switchover selection switching unit 531 and an abnormality detecting unit 532. It is noted that the coil connected to each of the receiving antennas 40a to 40h has the same electric properties as the coil L1.

The power switchover selection switching unit 531 is configured with a mechanical switch, a semiconductor switch, and the like. When a selection signal S2 for selecting the receiving antennas 40a to 40h to be supplied with power from the control unit 59 is inputted, the power switchover selection switching unit 531 selects the receiving antennas 40a to 40h as instructed by the selection signal S2 and supplies power only to the selected receiving antenna 40a to 40h.

When an abnormality occurs in the receiving antenna 40a to 40h to be supplied with power, the abnormality detecting unit 532 outputs, to the control unit 59, an abnormality signal indicating the occurrence of the abnormality in the receiving antenna 40a to 40h to be supplied with power.

The display unit 54 is configured with a display panel of the liquid crystal, the organic EL (Electro Luminescence), and the like. The display unit 54 displays various sorts of information such as the image corresponding to the image data taken by the capsule endoscope 3, the operation state of the receiving device 5, the patient information for the subject 2, the examination date and time, and so on.

The operating unit 55 is able to input the instruction signal such as for instructing the capturing period of the capsule endoscope 3 to be changed. In response to the instruction signal inputted from the operating unit 55, the signal processing circuit 51 sends the instruction signal to the transceiving circuit 50 and the transceiving circuit 50 modulates the instruction signal to transmit it from the receiving antennas 40a to 40h. The signals transmitted from the receiving antennas 40a to 40h are received by an antenna 39 and demodulated by the transceiving circuit 37, and the circuit board 36 operates for changing the capturing period, for example, in response to the instruction signal.

The storage unit 56 is configured with a semiconductor memory such as a flash memory, a RAM (Random Access Memory), and the like fixedly provided inside the receiving device 5. The storage unit 56 has theoretical electric field strength data 561 for the estimation process of the position and orientation, at which the image data has been taken, of the capsule endoscope 3 inside the subject 2. The theoretical electric field strength data 561 is the theoretical value data of the received electric field strength of the wireless signal received by each of the receiving antennas 40a to 40h depending on the position and orientation of the capsule endoscope 3 in the subject 2. Further, the storage unit 56 stores the image data taken by the capsule endoscope 3 and various sorts of information associated with that image data, such as the estimated position and orientation information of the capsule endoscope 3, the received electric field strength information, and the identification information for identifying the receiving antenna which has received the wireless signal, and so on. Further, the storage unit 56 stores various programs and the like that is executed by the receiving device 5. It is noted that the storage unit 56 may be provided with the function of a recording medium interface for storing the information to a recording medium such as a memory card and the like from the external unit while reading out the information stored in the recording medium.

The I/F unit 57 has the function as the communication interface and communicates bi-directionally with the image display device 6 via the cradle 6a.

The power supply unit 58 is configured with a battery that is removable from the receiving device 5 and a switch unit that switches a turn-on/off state. The power supply unit 58 supplies a driving power necessary for each component of the receiving device 5 in the turn-on state, while stops supplying the driving power to each component of the receiving device 5 in the turn-off state.

The control unit 59 is configured with a CPU (Central Processing Unit) and the like. The control unit 59 reads out the program from the storage unit 56 to execute it and transfers the instruction, the data, and so on to each unit of the receiving device 5 to control the operation of the receiving device 5 in an integrated manner. The control unit 59 has a selection control unit 591, an abnormality information adding unit 592, an electric field strength comparing unit 593, and a position determination unit 594.

The selection control unit 591 selects one receiving antenna 40 for receiving the wireless signal transmitted from the capsule endoscope 3 and controls to supply power only to the selected receiving antenna 40a to 40h. Specifically, the selection control unit 591 selects one receiving antenna 40 for receiving the wireless signal transmitted from the capsule endoscope 3 based on the received electric field strength of each of the receiving antennas 40a to 40h detected by the received electric field strength detecting unit 52, and controls to supply power only to the selected antenna 40a to 40h. The selection control unit 591 drives the antenna switchover selection switching unit 49 for every specified timing such as for every 100 msec, and sequentially selects one receiving antenna 40a to 40h for receiving the wireless signal from the receiving antennas 40a to 40h and causes the received electric field strength detecting unit 52 to detect the received electric field strength.

When the abnormality detecting unit 532 detects an abnormality at any one of the receiving antennas 40a to 40h, the abnormality information adding unit 592 adds, to the wireless signal received by the receiving antenna 40, abnormality information indicating the occurrence of the abnormality at any one of the receiving antennas 40a to 40h and outputs it to the storage unit 56. Specifically, the abnormality information adding unit 592 adds the abnormality information (flag) to the image data for which the signal processing circuit 51 has applied the signal processing to the wireless signal received by the receiving antennas 40a to 40h, and outputs it to the storage unit 56.

The electric field strength comparing unit 593 calculates the residual sum of squares between the received electric field strength of the wireless signal received by each of the receiving antennas 40a to 40h and the theoretical electric field strength stored in the storage unit 56 for each position and orientation within the subject 2 at which the capsule endoscope 3 can be present inside the subject 2. The electric field strength comparing unit 593 may calculate and compare the sum of the absolute residuals between the received electric field strength and the theoretical electric field strength in place of the residual sum of squares.

Based on the residual sum of squares or the sum of the absolute residuals calculated by the electric field strength comparing unit 593, the position determination unit 594 determines the position and orientation of the capsule endoscope 3 at which the image data has been taken. The position determination unit 594 determines the region and orientation having the smallest residual sum of squares as the position and orientation of the capsule endoscope 3 at which the image data has been taken.

In the first embodiment, the receiving device 5 has the storage unit 56 for storing the theoretical electric field strength data 561, the electric field strength comparing unit 593 for calculating the residual sum of squares of the received electric field strength and the theoretical electric field strength, and the position determination unit 594 for determining the position and orientation of the capsule endoscope 3 based on the residual sum of squares calculated by the electric field strength comparing unit 593, and calculates with these elements the position and orientation of the image data taken by the capsule endoscope 3. Described below in detail will be the estimation process of the position and orientation of the capsule endoscope 3 in the receiving device 5 of the first embodiment.

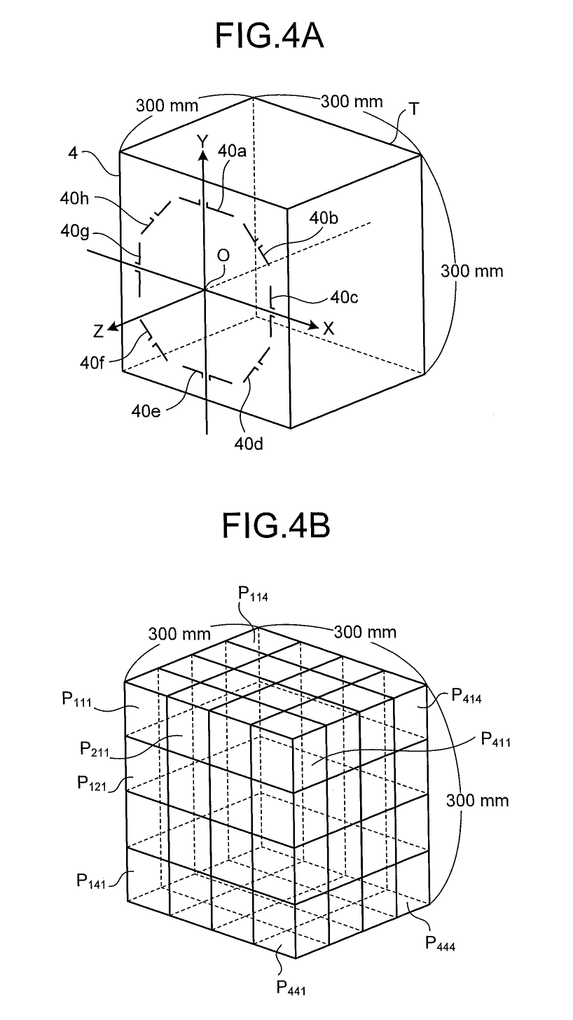

First, described will be the calculation process of the theoretical electric field strength data 561 to be pre-stored in the storage unit 56. A specified possible occurrence region T where the capsule endoscope 3 can be present within the subject 2 into which the capsule endoscope 3 is introduced is initially set according to the purpose of the examination, the diagnosis, and the like. This possible occurrence region T is set depending on the size of the body of the subject 2, which will be a region of a cube of 300 mm×300 mm×300 mm as illustrated in FIG. 4A, for example. The possible occurrence region T is set so that the sheet-shaped surface of the receiving antenna unit 4 matches one of the border planes. In the case illustrated in FIG. 4A, the receiving antenna unit 4 is provided on the XY plane that is one of the border planes of the possible occurrence region T.

The possible occurrence region of the capsule endoscope 3 is divided into a plurality of subregions according to the desired accuracy. FIG. 4B illustrates a case where it is divided into four regions in each axis direction with respect to the orthogonal coordinate system XYZ having three axes (X axis, Y axis, Z axis) that are parallel to any one of the edges of the possible occurrence region T and are orthogonal to each other, where the origin is assumed to the center of the border plane on which the receiving antenna unit 4 is located. In this case, the possible occurrence region T is divided into 64 (=4×4×4) subregions. The subregions are labeled with P₁₁₁, P₁₁₂, P₁₁₃, P₁₁₄, P₁₂₁, P₁₂₂, . . . , P₂₁₁, P₂₁₂, . . . , P₄₄₄. It is noted that, when the capsule endoscope 3 is present in a subregion Pᵢⱼₖ, it is assumed the capsule endoscope 3 being located at the center G_{xyz} of the subregion Pᵢⱼₖ.

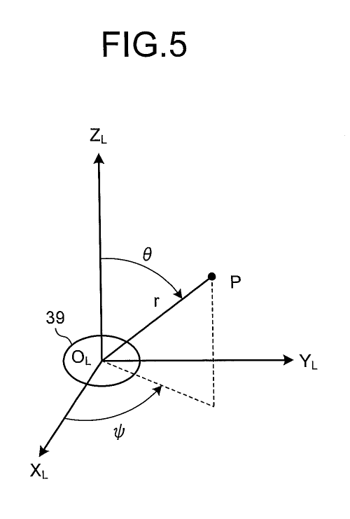

In the following description, as illustrated in FIG. 5, the center of gravity of the circle loop antenna 39 disposed in the capsule endoscope 3 is defined as the origin (O_{L}), and the orthogonal coordinate system X_{L}Y_{L}Z_{L} will be considered where the normal line direction of the opening plane of the circle loop is defined as the Z_{L} axis. In the orthogonal coordinate system X_{L}Y_{L}Z_{L}, the polar coordinate components of the electromagnetic field that is formed at a particular position P by the current flowing in the antenna 39 are represented by the following equations.

Hᵣ=(IS/2π)(jk/r²+1/r³)exp(−jkr)cos θ

H_{θ}=(IS/4π)(−k²/r+jk/r²+1/r³)exp(−jkr)sin θ

E_{ψ}=−(jωμIS/4π)(jk/r+1/r²)exp(−jkr)sin θ (1)

Here, Hᵣ and H_{θ} denote the magnetic field components, E_{ψ} denotes the electric field component, and I and S denote the current flowing in the antenna 39 and the region of the opening region of the circle loop of the antenna 39. Further, k=ω(∈μ)^{1/2} (∈ is the dielectric constant, μ is the magnetic permeability) denotes the number of the waves, and j represents the unit of the imaginary number. Here, in Equations (1), the term of r⁻¹ denotes the radiation electromagnetic field, the term of r⁻² denotes the induction electromagnetic field, and the term of r⁻³ denotes the static electromagnetic field.

When the frequency of the electromagnetic field generated by the antenna 39 disposed within the capsule endoscope 3 is high and the capsule endoscope 3 and each receiving antenna 40 (40a to 40h) attached to the body surface of the subject 2 are sufficiently distant, the component of the radiation electromagnetic field is the greatest in the electromagnetic field (the electromagnetic wave) reaching the receiving antenna 40 (40a to 40h). Thus, the components of the static electromagnetic field and the induction electromagnetic field are smaller than the component of the radiation electromagnetic field and thus can be ignored. Therefore, Equations (1) can be expressed as the following Equations (2).

Hᵣ=0

H_{θ}=(IS/4π)(−k²/r)exp(−jkr)sin θ

E_{ψ}=−(jωμIS/4π)(jk/r)exp(−jkr)sin θ (2)

Assuming that the receiving antenna 40 attached to the body surface of the subject 2 is the electric field detecting antenna for detecting the electric field, the necessary equation for detecting it in Equations (2) will be the electric field E. Therefore, the instantaneous value of the electric field can be derived by using the alternating current theory, that is, multiplying both sides of the electric field E_{ψ} of Equations (2) by exp(jωt) to extract the real number part.

$$\begin{matrix}{E}_{ψ} exp ( jω t ) = - ( j ωμ IS / 4 π ) ( j k / r ) exp ( - j kr ) sin θexp ( jω t ) \\ = ( ωμ ISk / 4 π r ) ( cos U + j sin U ) sin θ\end{matrix} \qquad ( 3 )$$

Here, U=ωt−kr. Then, the real number part of Equation (3) is extracted to have the following instantaneous value E′_{ψ} of the electric field.

E′_{ψ}=(ωμISk/4πr)cos U sin θ (4)

Further, when Equation (4) is represented in the orthogonal coordinate system X_{L}Y_{L}Z_{L}, the components E_{Lx}, E_{Ly}, and E_{Lz} will be as follows.

E_{Lx}=E′_{ψ} sin ψ=(ωμISk/4πr²)cos U·(−y_{L})

E_{Ly}=E′_{ψ} cos ψ=(ωμISk/4πr²)cos U·x_{L}

E_{Lz}=0 (5)

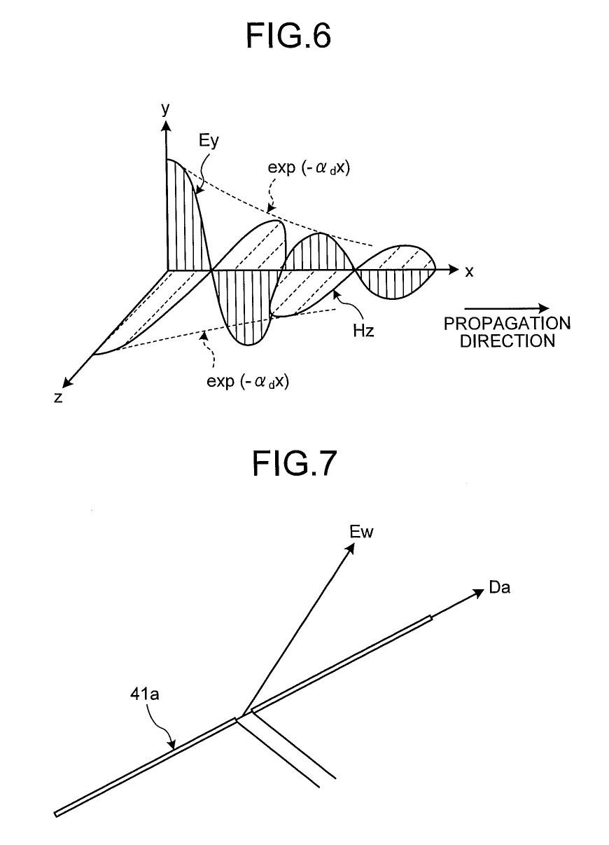

When the electromagnetic wave travels in the medium, the characteristics (the electric conductivity and the like) of the medium causes the energy of the electromagnetic wave to be absorbed in the medium through which the electromagnetic wave travels, as illustrated in FIG. 6. The electromagnetic wave attenuates exponentially at an attenuation factor α_{d} as it propagates in the x direction, which can be expressed by the following Equations (6).

Aᵣ=exp(−α_{d}x)

α_{d}=(ω²∈μ/2)^{1/2}[(1+κ²/(ω²∈²))^{1/2}−1]^{1/2} (6)

Here, ∈=∈ₒ∈ᵣ (∈ₒ: the dielectric constant of the vacuum, ∈ᵣ: the relative dielectric constant), μ=μₒμᵣ (μₒ: the magnetic permeability of the vacuum, μᵣ: the relative magnetic permeability), ω is the angular frequency, and κ is the electric conductivity.

Therefore, respective components E_{Lx}, E_{Ly}, and E_{Lz} of the orthogonal coordinate system X_{L}Y_{L}Z_{L} of the instantaneous value E_{L} of the electric field at the time when the characteristics in the organism is taken into consideration are as follows.

E_{Lx}=AᵣE′_{ψ} sin ψ=exp(−α_{d}r)(ωμISk/4πr²)cos U·(−y_{L})

E_{Ly}=AᵣE′_{ψ} cos ψ=exp(−α_{d}r)(ωμISk/4πr²)cos U·x_{L}

E_{Lz}=0 (7)

Further, in the coordinate system X_{L}Y_{L}Z_{L} with respect to the antenna 39 of the capsule endoscope 3, the equation for converting the position P (X_{L}, Y_{L}, Z_{L}) into the coordinate system XYZ whose origin is the center (◯ in FIG. 4A) of the receiving antenna unit 4 attached to the subject 2 is:

$$\begin{matrix}( \begin{matrix}{x}_{LP} \\ {y}_{LP} \\ {z}_{LP}\end{matrix} ) = {R}^{- 1} [ ( \begin{matrix}{x}_{WP} \\ {y}_{WP} \\ {z}_{WP}\end{matrix} ) - ( \begin{matrix}{x}_{WG} \\ {y}_{WG} \\ {z}_{WG}\end{matrix} ) ] \\ = ( \begin{matrix}{R}_{00} & {R}_{01} & {R}_{02} \\ {R}_{10} & {R}_{11} & {R}_{12} \\ {R}_{20} & {R}_{21} & {R}_{22}\end{matrix} ) [ ( \begin{matrix}{x}_{WP} \\ {y}_{WP} \\ {z}_{WP}\end{matrix} ) - ( \begin{matrix}{x}_{WG} \\ {y}_{WG} \\ {z}_{WG}\end{matrix} ) ]\end{matrix} \qquad ( 8 )$$

Here, (x_{WP}, y_{WP}, z_{WP}) and (x_{WG}, y_{WG}, z_{WG}) represent the position P in the coordinate system X_{W}Y_{W}Z_{W} and the position G of the antenna 39, respectively. Further, the right side R of Equation (8) represents the rotation matrix of the coordinate system X_{W}Y_{W}Z_{W} and the coordinate system X_{L}Y_{L}Z_{L}, and can be derived by the following equation.

$$( \begin{matrix}{R}_{00} & {R}_{10} & {R}_{20} \\ {R}_{01} & {R}_{11} & {R}_{21} \\ {R}_{02} & {R}_{12} & {R}_{22}\end{matrix} ) = ( \begin{matrix}cos α cos β & - sin α & cos α sin β \\ sin α cos β & cos α & sin α sin β \\ - sin β & 0 & cos β\end{matrix} ) \qquad ( 9 )$$

Here, α is the rotation angle around the Z axis and β is the rotation angle around the Y axis.

Therefore, the electric field E_{W} of some particular position P (x_{WP}, y_{WP}, z_{WP}) in the coordinate system X_{W}Y_{W}Z_{W} whose origin is the center (◯ in FIG. 4A) of the receiving antenna unit 4 attached to the subject 2 is:

$$\begin{matrix}Ew = ( \begin{matrix}{E}_{Wx} \\ {E}_{Wy} \\ {E}_{Wz}\end{matrix} ) \\ = R ( \begin{matrix}{E}_{Lx} \\ {E}_{Ly} \\ {E}_{Lz}\end{matrix} ) \\ = ( \begin{matrix}{R}_{00} & {R}_{10} & {R}_{20} \\ {R}_{01} & {R}_{11} & {R}_{21} \\ {R}_{02} & {R}_{12} & {R}_{22}\end{matrix} ) ( \begin{matrix}{E}_{Lx} \\ {E}_{Ly} \\ {E}_{Lz}\end{matrix} )\end{matrix} \qquad ( 10 )$$

Then, Equations (7) to (9) are substituted in Equation (10) resulting in the following equation of the electric field E_{W} (11).

$$( \begin{matrix}{E}_{Wx} \\ {E}_{Wy} \\ {E}_{Wz}\end{matrix} ) = \frac{{k}_{1}}{{r}^{2}} {e}^{- {a}_{d} r} ( \begin{matrix}0 & ( {z}_{WP} - {z}_{WG} ) & - ( {y}_{WP} - {y}_{WG} ) \\ - ( {z}_{WP} - {z}_{WG} ) & 0 & ( {x}_{WP} - {x}_{WG} ) \\ ( {y}_{WP} - {y}_{WG} ) & - ( {x}_{WP} - {x}_{WG} ) & 0\end{matrix} ) ( \begin{matrix}{g}_{x} \\ {g}_{y} \\ {g}_{z}\end{matrix} ) \qquad ( 11 )$$

Here, k₁ is a constant, the vector (gₓ, g_{y}, g_{z}) denotes the orientation g of the antenna 39. In the first embodiment, the orientation (gₓ, g_{y}, g_{z}) of the antenna 39 is preset together with the position of the capsule endoscope 3 to calculate the theoretical electric field strength of each receiving antenna 40 when the capsule endoscope 3 is located at a specified region and at a specified orientation. The orientation of the antenna 39 may be set by 1° pitch from the horizontal axis and the vertical axis, for example, according to the desired accuracy.

Further, the electromotive force Vₜₐ detected when the electric field E_{W} generated by the antenna 39 is received by the receiving antenna 40a of the receiving antenna unit 4 can be calculated by using the following equation in a use of the inner product of the electric field E_{W} and the vector Dₐ=(Dₓₐ, D_{ya}, D_{za}) (see FIG. 7) representing the orientation of the receiving antenna 40a (the antenna unit 41a) of the receiving antenna unit 4 in the coordinate system with respect to the subject 2.

Vₜₐ=k₂(E_{Wx}Dₓₐ+E_{Wy}D_{ya}+E_{Wz}D_{za}) (12)

Here, k₂ is a constant. For each of the plurality of receiving antennas of the receiving antenna unit 4 provided to the body of the subject 2, the similar is applied to derive the electromotive forces V_{tb}, . . . , Vₜₕ when the receptions are made at the receiving antenna 40b to the receiving antenna 40h.

In such a manner as described above, the theoretical electric field strength Vₜᵢ received by each receiving antenna 40 is calculated and stored in the storage unit 56 as the theoretical electric field strength data 561 for each center position G in the divided region.

The electric field strength comparing unit 593 calculates the residual sum of squares between the received electric strength received by each receiving antenna 40 and the theoretical electric field strength stored in the storage unit 56 as the theoretical electric field strength data 561 calculated as described above for each orientation g of the antenna 39 for the center position G of each region where the capsule endoscope 3 can be present. Assuming that the electric field strength Vₘᵢ (“i” represents the number of the receiving antenna, i=a to h in the present embodiment) received by the receiving antenna 40, the residual sum of squares S can be calculated by the following equation.

$$\begin{matrix}S = {\sum }_{i = a}^{h} {( {V}_{ti} - {V}_{m i} )}^{2} \\ = {( {V}_{ta} - {V}_{ma} )}^{2} + {( {V}_{tb} - {V}_{mb} )}^{2} + ⋯ + {( {V}_{th} - {V}_{mh} )}^{2}\end{matrix} \qquad ( 13 )$$

In the first embodiment, as described above, the electric field strength comparing unit 593 calculates the residual sum of squares between the received electric field strength Vₘᵢ received by each receiving antenna 40 and the theoretical electric field strength Vₜᵢ stored in the storage unit 56 as the theoretical electric field strength data 561 calculated as described above for each orientation g of the antenna 39 for the center position G of each region where the capsule endoscope 3 can be present, so that the same number of the CPUs as the total number of the center positions G for estimation (alternatively, it may be the factor of the total number of the center positions G for estimation, or the number less than or equal to the factor) can be used as the electric field strength comparing unit 593 at the same time for the estimation process, which allows for the faster estimation process of the position and orientation of the capsule endoscope 3.

The position determination unit 594 determines, as the position and orientation of the capsule endoscope 3, the center position G of the capsule endoscope 3 and the orientation g of the antenna 39 having the smallest one of the residual sum of squares S calculated by the electric field strength comparing unit 593 as described above.

In the first embodiment, the region where the capsule endoscope 3 can be present is divided into a plurality of subregions and the theoretical electric field strength Vₜᵢ depending on the orientation of the capsule endoscope 3 for each divided region is stored in advance, so that the processing load for calculating the theoretical electric field strength Vₜᵢ can be reduced. Further, the position and orientation of the capsule endoscope 3 at which the image data has been taken is determined based on the numeral value that can be obtained by simple calculation process of the residual sum of squares between the stored theoretical electric field strength Vₜᵢ and the received electric field Vₘᵢ that has been actually received by the receiving antenna 40, which allows for the faster position estimation process.

Furthermore, in the first embodiment, the sheet-shaped receiving antenna unit 4 on which a plurality of receiving antennas 40 are provided, so that it is not necessary to adjust the position of each receiving antenna 40 for every examination. Moreover, the receiving antenna unit 4 on which the position of each receiving antenna 40 is determined in advance is used, so that the problem of reduction in accuracy in the estimation process of the position and orientation of the capsule endoscope 3 that would otherwise be caused by the position shift of each receiving antenna 40 can be advantageously avoided.

Although the position detecting apparatus for performing the estimation process of the position and orientation of the capsule endoscope 3 has been described in the first embodiment, the apparatus may estimate either one of the position and the orientation of the capsule endoscope 3. Further, the receiving device 5 includes the storage unit 56 for storing the theoretical electric field strength data 561, the electric field strength comparing unit 593, and the position determination unit 594, and the position and orientation of the capsule endoscope 3 is estimated in the receiving device 5 in the first embodiment. However, the image display device 6 of the capsule endoscope system 1 may store the theoretical electric field strength, include the electric field strength comparing unit and the position determination unit, receive the image data transmitted from the receiving device, and perform the calculation similarly to the above to estimate the position and orientation of the capsule endoscope at which the image data has been taken.

In the first embodiment, the estimation process of the position and orientation of the capsule endoscope is made by calculating in parallel the residual sum of squares between the theoretical electric field strength and the received electric field strength with respect to all the set directions (alternatively, the reduced directions) in all the regions (alternatively, the reduced regions for simplification) divided as the position P where the capsule endoscope can be present. In contrast, a second embodiment divides it into hierarchies with two or more steps and determines the position and orientation of the capsule endoscope at which the image data has been taken.

In the second embodiment below, described will be a case where the estimation of the position and orientation of the capsule endoscope is divided into two steps. First, similarly to the first embodiment, the possible occurrence region T where the capsule endoscope 3 can be present is set in the subject 2 into which the capsule endoscope 3 is introduced, according to the purpose of examination, diagnosis, and the like. For example, it may be the region of a cube of 300 mm×300 mm×300 mm as illustrated in FIG. 4A. The possible occurrence region T is set so that the sheet-shaped surface of the receiving antenna unit 4 matches one of the border planes. In the case illustrated in FIG. 4A, the receiving antenna unit 4 is provided on the XY plane that is one of the border planes of the possible occurrence region T.

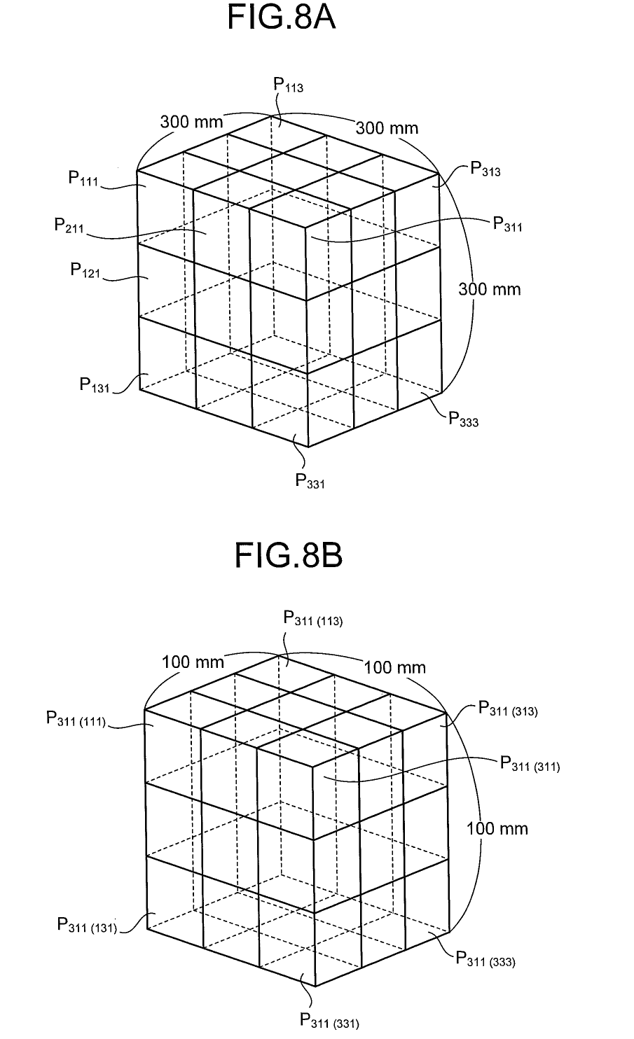

The possible occurrence region T of the capsule endoscope 3 is divided into a plurality of subregions according to the desired accuracy. FIG. 8A illustrates a case where it is divided into three regions in each axis direction with respect to the orthogonal coordinate system XYZ having three axes (X axis, Y axis, Z axis) that are parallel to any one of the edges of the possible occurrence region T and are orthogonal to each other, where the origin is assumed to the center of the border plane on which the receiving antenna unit 4 is located. In this case, the possible occurrence region T is divided into 27 (=3×3×3) subregions. The subregions are labeled with P₁₁₁, P₁₁₂, P₁₁₃, P₁₂₁, P₁₂₂, . . . , P₁₃₃, P₂₁₁, P₂₁₂, . . . , P₃₃₃. It is noted that, when the capsule endoscope 3 is present in a subregion Pᵢⱼₖ, it is assumed to be located at the center G_{xyz} of the subregion Pᵢⱼₖ.

FIG. 8A is a schematic diagram in which the possible occurrence region T of the capsule endoscope 3 is divided into three regions in each of the x, y, and z axis directions. FIG. 8B is a schematic diagram in which one of the divided regions in FIG. 8A is further divided into three regions in each of the x, y, and z axis directions.

The position detecting apparatus performs, as a first estimation step, an estimation process of the position and orientation for the regions resulted after the possible occurrence region T (300 mm×300 mm×300 mm) where the capsule endoscope 3 can be present within the subject 2 is divided into three regions for each of the x, y, and z axis directions. As illustrated in FIG. 8A, for each center position G of the regions labeled with P₁₁₁, P₁₁₂, P₁₁₃, P₁₂₁, P₁₂₂, . . . , P₁₃₃, P₂₁₁, P₂₁₂, . . . , P₃₃₃, the electric field strength comparing unit 593 calculates the residual sum of squares between the received electric field strength received by each receiving antenna 40 and the theoretical electric field strength stored as the theoretical electric field strength data 561 in the storage unit 56 for each orientation g of the antenna 39. Since a purpose of the first estimation step is to approximately specify the position and orientation, the orientation g of the antenna 39 in the estimation process is reduced to one, or is significantly reduced (for example, by 10° pitch from the horizontal axis and the vertical axis).

The position determination unit 594 determines, as the position and orientation of the first step, the center position G of the region of the capsule endoscope 3 and the orientation g of the antenna 39 which has the smallest one of the residual sums of squares S calculated by the electric field strength comparing unit 593.

As a second estimation step, another estimation process of the position and orientation is made for the regions resulted after the region including the position G of the capsule endoscope 3 determined by the position determination unit 594 at the first estimation step is further divided into three regions for each of the x, y, and z axis directions (27 regions in total).

For example, as the first estimation step, it is assumed that the position determination unit 594 selects the position P₃₁₁ illustrated in FIG. 8A as the position and orientation of the capsule endoscope 3. FIG. 8B illustrates that the position P₃₁₁ is divided into three regions for each axis direction with respect to the orthogonal coordinate system XYZ having three axes (X axis, Y axis, Z axis) that are parallel to one of the edges of the position P₃₁₁ and orthogonal to each other. In this case, the position P₃₁₁ is further divided into 27 (=3×3×3) subregions. The subregions are labeled with P₃₁₁₍₁₁₁₎, P₃₁₁₍₁₁₂₎, P₃₁₁₍₁₁₃₎, P₃₁₁₍₁₂₁₎, P₃₁₁₍₁₂₂₎, . . . , P₃₁₁₍₁₃₃₎, P₃₁₁₍₂₁₁₎, P₃₁₁₍₂₁₂₎, . . . , P₃₁₁₍₃₃₃₎. The electric field strength comparing unit 593 calculates the residual sum of squares between the received electric field strength received by each receiving antenna 40 and the theoretical electric field strength stored as the theoretical electric field strength data 561 in the storage unit 56 for each orientation g of the antenna 39. In the second estimation step, the orientation g of the antenna 39 is estimated according to the desired accuracy. For example, it is applied for all the directions by 1° pitch from the horizontal axis and the vertical axis.

The position determination unit 594 determines, as the final position and orientation of the capsule endoscope 3 at which the image data has been taken, the position P_{xyz(xyz)} of the capsule endoscope 3 and the orientation gₙ (gₙₓ, g_{ny}, g_{nz}) of the antenna 39 which has the smallest one of the residual sums of squares S_{xyz(xyz)n} calculated by the electric field strength comparing unit 593.

It is noted that, as described above, even if the estimation process is performed with the division of two steps of hierarchy, it is necessary for the storage unit 56 to store the theoretical electric field strength data 561 received by each receiving antenna 40 by each orientation g of the antenna 39 (by 1° pitch from the horizontal axis and the vertical axis) in the region position P_{xyz(xyz)} resulted after the possible occurrence region T (300 mm×300 mm×300 mm) where the capsule endoscope 3 can be present is divided into nine regions for each of the x, y, and z axis directions.

In the second embodiment, the position and orientation of the capsule endoscope 3 at which the image data has been taken is divided into two steps of hierarchy, the approximate position and orientation of the capsule endoscope 3 is determined in the first estimation step, and the second estimation process is further applied to the limited regions, so that the amount of processing can be reduced compared to the case where the estimation process is applied at the same time for the regions of a similar size. This allows for much faster position estimation process.

It is noted that, although the example in which the position and orientation of the capsule endoscope 3 is divided into two steps of hierarchy for the estimation process has been described in the second embodiment, the estimation process may be performed with the division of three or more steps of hierarchy, because two or more steps can allow for the reduced amount of the estimation processing. It is noted that, regarding the orientation of the capsule endoscope 3 (the orientation of the antenna 39), the estimation process may be performed at a desired accuracy at the first step, for example, by 1° pitch from the horizontal axis and the vertical axis for all the directions.

When the position and orientation of the capsule endoscope 3 at which the image data has been taken is determined as described in the first and second embodiments, there is a case where the correct position and orientation cannot be estimated due to the positional error of the receiving antenna, the noise, and so on. In a third embodiment, the position and direction of the capsule endoscope 3 is estimated based on the estimated position information of the image data taken at a previous timing and a subsequent timing.

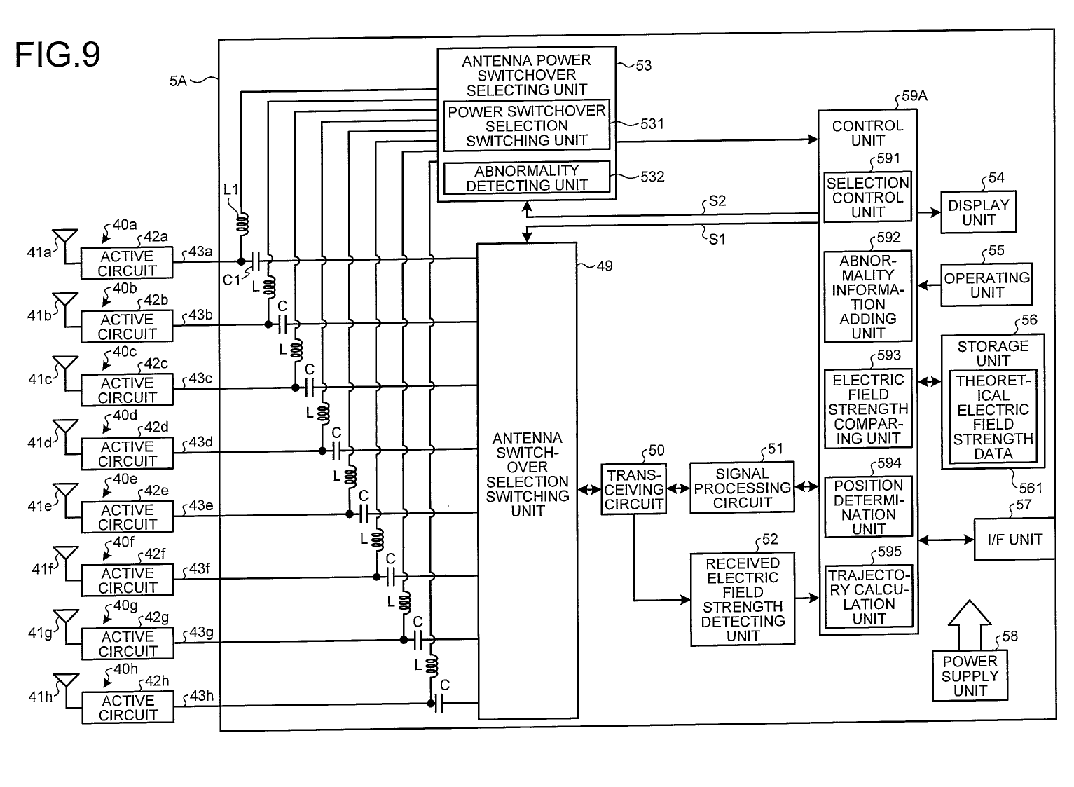

FIG. 9 is a block diagram illustrating a configuration of a receiving device 5A according to the third embodiment. The receiving device 5A includes a trajectory calculation unit 595 for calculating the distances between a plurality of candidate positions selected as the position of the capsule endoscope 3 by the position determination unit 594 and the previous and subsequent candidate positions, determining whether the distances are less than or equal to a specified value, and calculating the movement trajectory (path) of the capsule endoscope within the subject 2 using the distances which satisfy the condition.

In general, the movement of the capsule endoscope 3 is relatively small and the photographing interval is quite short. Thus, the capturing position of the image data that has been taken at a particular time is often substantially the same as or close to the capturing position of the image data that have been taken previously and subsequently with respect to the time when that image data has been taken.

In the third embodiment, the electric field strength comparing unit 593 calculates the residual sum of squares between the theoretical electric field strength and the received electric field strength similarly to the first and second embodiments. The position determination unit 594 selects the smallest i residual sums of squares (i is an arbitrary number, the present embodiment is described as i=3) for the candidates of the position and orientation of the capsule endoscope at which the image data has been taken, and stores them into the storage unit 56. The trajectory calculation unit 595 calculates the movement trajectory of the capsule endoscope 3 within the subject 2 in taking into consideration of the estimated distance to a plurality of candidate positions of the image data taken at a previous timing and a subsequent timing. The position determination unit 594 determines the optimum position and orientation of the capsule endoscope based on the trajectory calculated by the trajectory calculation unit 595.

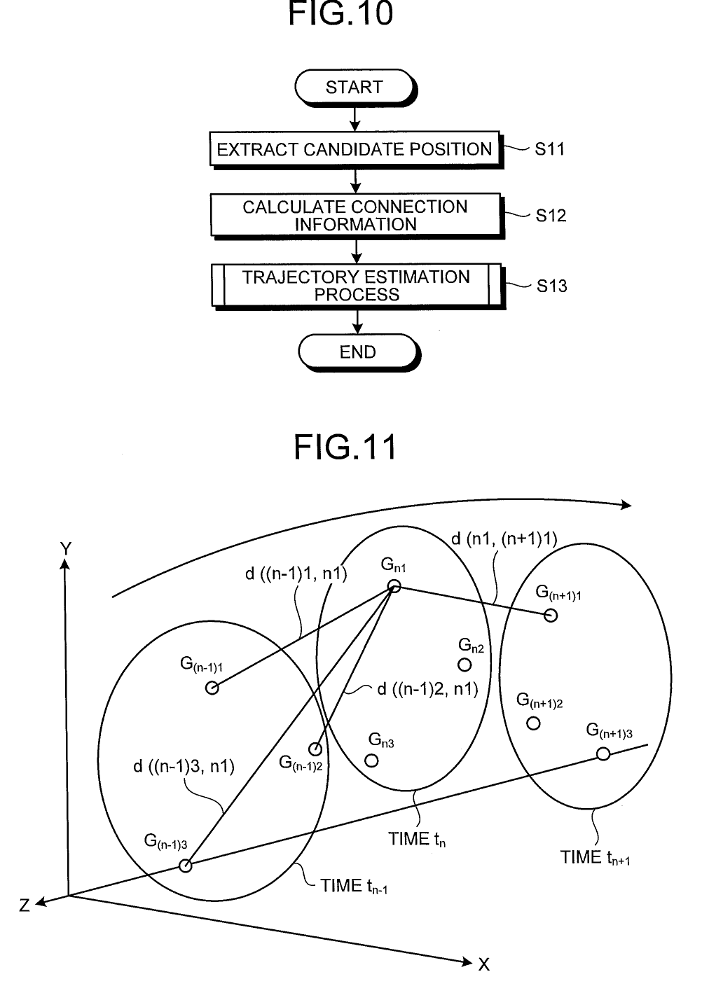

FIG. 10 is a flowchart illustrating the outline of the trajectory calculation process made by the trajectory calculation unit 595. The position determination unit 594 extracts the candidate position of the capturing position for each time calculated by the electric field strength comparing unit 593 (step S11). Specifically, the position determination unit 594 extracts, as the capturing position of the image data taken at each time, the three smallest residual sums of squares between the theoretical electric field strength and the received electric field strength in each position and orientation. Hereafter, the capturing position of the image data Dₘ taken at the time tₘ (m=1, 2, . . . , n, . . . , N) is assumed to be Gₘᵢ (i=1, 2, 3). FIG. 11 is a view schematically illustrating the candidate positions extracted at m=n−1, n, n+1.

Subsequently, the trajectory calculation unit 595 calculates the information of the connection to the candidate positions in the previous and subsequent image data with respect to the extracted candidate positions Gₘᵢ (step S12). Here, the receiving device 5A pre-stores in the storage unit 56 the distance r_{d} that the capsule endoscope 3 can move inside the subject 2 within one time interval depending on the time interval for the position estimation of the capsule endoscope 3.

The trajectory calculation unit 595 calculates the distances d((m−1)i, mj) between the candidate positions G₍ₘ₋₁₎ᵢ of the image data Dₘ₋₁, Dₘ at the neighboring time intervals tₘ₋₁, tₘ and the Gₘᵢ for all the combinations (m=2, . . . , N; i, j=1, 2, 3), and compares the calculated distances with the movable distance r_{d}. As a result of the comparison, the candidate position G₍ₘ₋₁₎ⱼ providing the distance d((m−1)i, mj) which is smaller than the movable distance r_{d} and is the smallest is stored in the storage unit 56 as the connection information of the candidate position Gₘᵢ. It is noted that, if all the distances d((m−1)i, mj) are larger than the movable distance r_{d} for a particular m, the trajectory calculation unit 595 does not store the connection information of the candidate position Qₘᵢ.

Subsequently, the trajectory calculation unit 595 uses each candidate position Gₘᵢ and the connection information of the candidate position to estimate the trajectory of the capsule endoscope 3 (step S13).

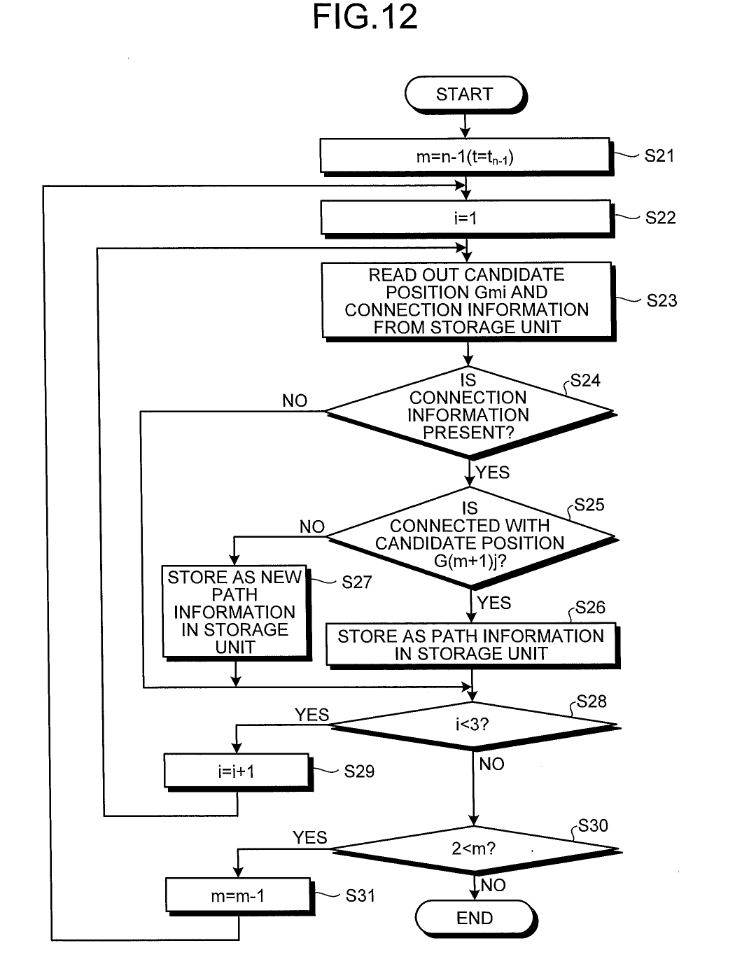

FIG. 12 is a flowchart illustrating an outline of the trajectory estimation process. In FIG. 12, the trajectory calculation unit 595 sets the time tₘ₋₁ that is immediately before the last time tₘ (step S21).

Then, the trajectory calculation unit 595 sets the parameter, which indicates the label of the candidate position at the time tₘ, to the initial value 1 (step S22).

Subsequently, the trajectory calculation unit 595 reads out the connection information of the candidate position Gₘᵢ from the storage unit 56 at the time tₘ (step S23).

If there is connection information G₍ₘ₋₁₎ⱼ in the candidate positions Gₘᵢ (Yes in step S24), the trajectory calculation unit 595 determines whether or not the candidate position Gₘᵢ is connected to the candidate position G₍ₘ₊₁₎ⱼ at the time tₘ₊₁ (step S25). If the candidate position Gₘᵢ is connected to any one of the candidate positions G₍ₘ₊₁₎ⱼ at the time tₘ₊₁ (Yes in step S25), in other words, if the candidate position Gₘᵢ is the connection information of any one of the candidate positions G₍ₘ₊₁₎ⱼ at the time tₘ₊₁, the trajectory calculation unit 595 stores the connected path information in the storage unit 56 (step S26).

On the other hand, if the candidate position Gₘᵢ is not connected to any one of the candidate positions G₍ₘ₊₁₎ⱼ at the time tₘ₊₁ (No in step S25), in other words, if the candidate position Gₘᵢ is not the connection information of any one of the candidate positions G₍ₘ₊₁₎ⱼ at the time tₘ₊₁, the trajectory calculation unit 595 stores new path information (the information indicating that the path is disconnected between the time tₘ and the time tₘ₊₁) in the storage unit 56 (step S27).

After step S26 or S27, if the parameter i is less than three (Yes in step S28), the trajectory calculation unit 595 increments i by one to have i+1 (step S29) and returns to step S23.

After step S26 or S27, if the parameter i is not less than three (No in step S28), if the time parameter m is m>2 (Yes in step S30), the trajectory calculation unit 595 decrements m by one to have m−1 (step S31) and returns to step S22. On the other hand, if m≦2 (No in step S30), the trajectory calculation unit 595 finishes the trajectory estimation process (step S13 of FIG. 10).

In such a way, the trajectory calculation unit 595 calculates the trajectory and estimates the position of the capsule endoscope 3 for each time.

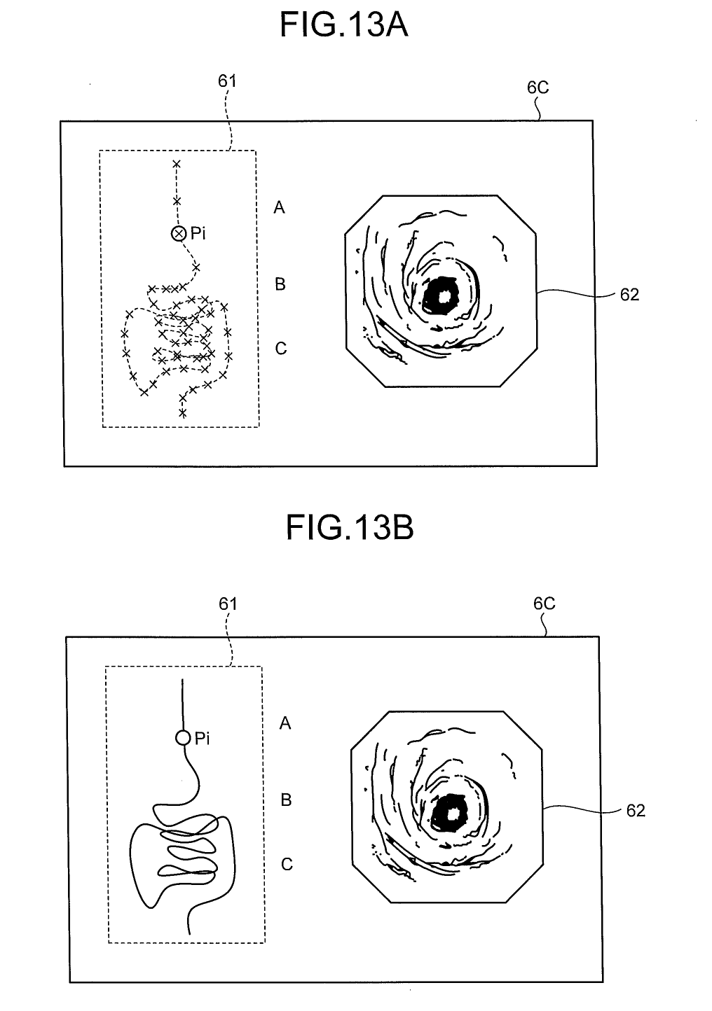

FIG. 13A and FIG. 13B are display examples on the monitor unit 6c of the image display device 6 that shows the trajectory of the capsule endoscope 3 within the subject 2 calculated by the receiving device 5A of the third embodiment. As illustrated in FIG. 13A, the monitor unit 6c includes a sub image region 61 in which the capturing positions of the capsule endoscope 3 within the subject 2 are connected by straight lines and the movement trajectory of the capsule endoscope 3 within the subject 2 is indicated, and a main image display region 62 in which the image data taken by the capsule endoscope 3 is displayed.

Further, the characters A, B, and C in the right of the sub image region 61 indicate the approximate positions of the organs in the body cavity. Specifically, the character A indicates the esophagus, the character B indicates the small intestine, and the character C indicates the large intestine. Further, the position Pᵢ indicates the position estimated as the capturing position of the image data which is displayed in the main image display region 62. Besides FIG. 13A in which the estimated capturing positions Pi are connected by the straight lines so that these are indicated as the trajectory, other display as illustrated in FIG. 13B, for example, may be employed in which the interpolation process such as the spline interpolation is applied to the neighboring capturing positions so that the capturing positions of the estimated capsule endoscope 3 are connected by smooth curves.

In the third embodiment, the position and orientation of the capsule endoscope 3 can be estimated without being affected by the noise and the like, so that the more accurate position and orientation of the capsule endoscope 3 can be derived. Further, the position and orientation of the capsule endoscope 3 is estimated and the movement trajectory of the capsule endoscope 3 inside the subject 2 is displayed on the image display device 6, so that it can be easily determined at which position in the body cavity the captured image has been taken, which allows for the efficient diagnosis. Further, there is a possibility that the obtained image is a lesion region, and when the further detailed endoscope examination is needed for that part, its position can be estimated in a high accuracy and thus the part can be approached smoothly in a short time, which allows for the efficient reexamination, treatment, and the like.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

Further, the above-described embodiments are mere examples for implementing the present invention and thus the present invention is not limited to them, the various modification according to the specification and the like is within the scope of the present invention, and it is clear from the above descriptions that other various embodiments are possible within the scope of the present invention.

1. A position detecting apparatus of a capsule endoscope comprising:
a receiving antenna unit for receiving, by a plurality of receiving antennas, a wireless signal transmitted from a capsule endoscope within a subject;
a storage unit for storing, in advance, information indicating a first position of the capsule endoscope within the subject and information indicating a first theoretical electric field strength of the wireless signal received by each of the antennas depending on the first position, in such a manner that the information of the first position is associated with the information of the first theoretical electric field strength, and for storing, in advance, information indicating a second position of the capsule endoscope within the subject and information indicating a second theoretical electric field strength of the wireless signal received by each of the antennas depending on the second position, in such a manner that the information of the second position is associated with the information of the second theoretical electric field strength;
an electric field strength comparing unit for comparing a received electric field strength of the wireless signal received by each of the receiving antennas with the first theoretical electric field strength and for comparing the received electric field strength with the second theoretical electric field strength; and
a position determination unit for determining either one of the first position and the second position, as a position of the capsule endoscope where image data has been taken, based on a comparison result by the electric field strength comparing unit.

2. The position detecting apparatus of the capsule endoscope according to claim 1, wherein the storage unit stores a theoretical electric field strength according to an orientation of the capsule endoscope for each of a plurality of subregions into which a region within the subject where the capsule endoscope can be present is divided.

3. The position detecting apparatus of the capsule endoscope according to claim 2, wherein
the electric field strength comparing unit calculates a residual sum of squares between the theoretical electric field strength stored in the storage unit and the received electric field strength for each of the subregions and for each orientation, and
the position determination unit determines, from a combination of an orientation and region having the smallest residual sum of squares, a position or a position and orientation of the capsule endoscope where the image data has been taken.

4. The position detecting apparatus of the capsule endoscope according to claim 3, wherein
the electric field strength comparing unit calculates the residual sum of squares according to the subregions divided into at least two or more steps of hierarchy and the orientation, for each divided hierarchy, and
the position determination unit limits, for each hierarchy, the region where the capsule endoscope is present, based on the comparison result by the electric field strength comparing unit, and determines the position and orientation of the capsule endoscope where the image data has been taken.

5. The position detecting apparatus of the capsule endoscope according to claim 3, wherein
the electric field strength comparing unit extracts, for each image data, a specified number of regions and orientations in ascending order of the residual sums of squares, as a candidate of the position and orientation of the capsule endoscope, and
based on a distance between candidate positions and/or the residual sum of squares of the image data for a previous timing and a subsequent timing, the position determination unit determines the position or the position and orientation of the capsule endoscope where each image data has been taken.

6. The position detecting apparatus of the capsule endoscope according to claim 1, wherein the receiving antenna unit has a sheet shape on which the plurality of receiving antennas are provided.

7. The position detecting apparatus of the capsule endoscope according to claim 1, further comprising a trajectory calculation unit for calculating a trajectory of the capsule endoscope based on the position of the capsule endoscope determined by the position determination unit.

8. A capsule endoscope system comprising:
a capsule endoscope for obtaining image data of an inside of a subject;
the position detecting apparatus according to claim 1 for receiving the image data transmitted from the capsule endoscope and estimating a position and orientation of the capsule endoscope where the received image data has been taken; and
an image display unit for obtaining the image data and position information of the image data from the receiving antenna and the position detecting apparatus and for displaying the obtained image data and position information.

9. The capsule endoscope system according to claim 8, wherein
the position detecting apparatus includes a trajectory calculation unit for calculating a trajectory of the capsule endoscope based on the position of the capsule endoscope determined by the position determination unit, and
the image display unit displays the image data and the trajectory in the subject of the capsule endoscope calculated by the trajectory calculation unit.
